(19) United States
(12) Patent Application Publication
(10) Pub. No.: US 2006/0166966 A1
(43) Pub. Date: Jul. 27, 2006

(54) 4-amino-azepan-3-one compounds as cathepsin k inhibitors useful in the treatment of osteoporosis

(76) Inventor: Cameron Black, Baie d'Urfe (CA)

Correspondence Address: MERCK AND CO., INC, P O BOX 2000, RAHWAY, NJ, 07065-0907, US

(21) Appl. No.: 10/530,250

(22) PCT Filed: Oct. 7, 2003
(86) PCT No.: PCT/CA03/01551

§ 371 Date: Apr. 5, 2005

Related U.S. Application Data
(60) Provisional application No. 60/416,892, filed on Oct. 8, 2002.

(51) Int. Cl. A61K 31/55; C07D 223/12

(52) U.S. Cl. 514/212.03; 540/527

(57) ABSTRACT

This invention relates to compounds of formula (I) which are cysteine protease inhibitors, in particular, inhibitors of cathepsins K, L, S and B. These compounds are useful for treating diseases in which inhibition of bone resorption is indicated, such as osteoporosis.

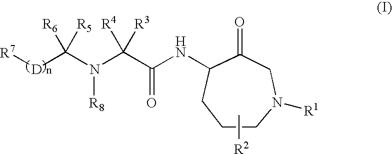

### BACKGROUND OF THE INVENTION

A variety of disorders in humans and other mammals involve or are associated with abnormal bone resorption. Such disorders include, but are not limited to, osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, metastatic bone disease, hypercalcemia of malignancy, and multiple myeloma. One of the most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Osteoporotic fractures are a major cause of morbidity and mortality in the elderly population. As many as 50% of women and a third of men will experience an osteoporotic fracture. A large segment of the older population already has low bone density and a high risk of fractures. There is a significant need to both prevent and treat osteoporosis and other conditions associated with bone resorption. Because osteoporosis, as well as other disorders associated with bone loss, are generally chronic conditions, it is believed that appropriate therapy will typically require chronic treatment.

Osteoporosis is characterized by progressive loss of bone architecture and mineralization leading to the loss in bone strength and an increased fracture rate. The skeleton is constantly being remodeled by a balance between osteoblasts that lay down new bone and osteoclasts that breakdown, or resorb, bone. In some disease conditions and advancing age the balance between bone formation and resorption is disrupted; bone is removed at a faster rate. Such a prolonged imbalance of resorption over formation leads to weaker bone structure and a higher risk of fractures.

Bone resorption is primarily performed by osteoclasts, which are multinuclear giant cells. Osteoclasts resorb bone by forming an initial cellular attachment to bone tissue, followed by the formation of an extracellular compartment or lacunae. The lacunae are maintained at a low pH by a proton-ATP pump. The acidified environment in the lacunae allows for initial demineralization of bone followed by the degradation of bone proteins or collagen by proteases such as cysteine proteases. See Delaisse, J. M. et al., 1980, Biochem J 192:365-368; Delaisse, J. et al., 1984, Biochem Biophys Res Commun: 441-447; Delaisse, J. M. et al., 1987, Bone 8:305-313, which are hereby incorporated by reference in their entirety. Collagen constitutes 95% of the organic matrix of bone. Therefore, proteases involved in collagen degradation are an essential component of bone turnover, and as a consequence, the development and progression of osteoporosis.

Cathepsins belong to the papain superfamily of cysteine proteases. These proteases function in the normal physiological as well as pathological degradation of connective tissue. Cathepsins play a major role in intracellular protein degradation and turnover and remodeling. To date, a number of cathepsin have been identified and sequenced from a number of sources. These cathepsins are naturally found in a wide variety of tissues. For example, cathepsin B, F, H, L, K, S, W, and Z have been cloned. Cathepsin K (which is also known by the abbreviation cat K) is also known as cathepsin O and cathepsin O2. See PCT Application WO 96/13523, Khepri Pharmaceuticals, Inc., published May 9, 1996, which is hereby incorporated by reference in its entirety. Cathepsin L is implicated in normal lysosomal proteolysis as well as several diseases states, including, but not limited to, metastasis of melanomas. Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis; allergic disorders, including, but not limited to asthma; and allogenic immunbe responses, including, but not limited to, rejection of organ transplants or tissue grafts. Increased Cathepsin B levels and redistribution of the enzyme are found in tumors, suggesting a role in tumor invasion and matastasis. In addition, aberrant Cathpsin B activity is implicated in such disease states as rheumatoid arthritis, osteoarthritis, pneumocystisis carinii, acute pancreatitis, inflammatory airway disease and bone and joint disorders.

Cysteine protease inhibitors such as E-64 (trans-epoxysuccinyl-L-leucylamide-(4-guanidino)butane) are known to be effective in inhibiting bone resorption. See Delaisse, J. M. et al., 1987, Bone 8:305-313, which is hereby incorporated by reference in its entirety. Recently, cathepsin K was cloned and found specifically expressed in osteoclasts See Tezuka, K. et al., 1994, J Biol Chem 269:1106-1109; Shi, G. P. et al., 1995, FEBS Lett 357:129-134; Bromme, D. and Okamoto, K., 1995, Biol Chem Hoppe Seyler 376:379-384; Bromme, D. et al., 1996, J Biol Chem 271:2126-2132; Drake, F. H. et al., 1996, J Biol Chem 271:12511-12516, which are hereby incorporated by reference in their entirety. Concurrent to the cloning, the autosomal recessive disorder, pycnodysostosis, characterized by an osteopetrotic phenotype with a decrease in bone resorption, was mapped to mutations present in the cathepsin K gene. To date, all mutations identified in the cathepsin K gene are known to result in inactive protein. See Gelb, B. D. et al., 1996, Science 273:1236-1238; Johnson, M. R. et al., 1996, Genome Res 6:1050-1055, which are hereby incorporated by reference in their entirety. Therefore, it appears that cathepsin K is involved in osteoclast mediated bone resorption.

Cathepsin K is synthesized as a 37 kDa pre-pro enzyme, which is localized to the lysosomal compartment and where it is presumably autoactivated to the mature 27 kDa enzyme at low pH. See McQueney, M. S. et al., 1997, J Biol Chem 272:13955-13960; littlewood-Evans, A. et al., 1997, Bone 20:81-86, which are hereby incorporated by reference in their entirety. Cathepsin K is most closely related to cathepsin S having 56% sequence identity at the amino acid level. The S₂P₂ substrate specificity of cathepsin K is similar to that of cathepsin S with a preference in the P1 and P2 positions for a positively charged residue such as arginine, and a hydrophobic residue such as phenylalanine or leucine, respectively. See Bromme, D. et al., 1996, J Biol Chem 271: 2126-2132; Bossard, M. J. et al., 1996, J Biol Chem 271:12517-12524, which are hereby incorporated by reference in their entirety. Cathepsin K is active at a broad pH range with significant activity between pH 4-8, thus allowing for good catalytic activity in the resorption lacunae of osteoclasts where the pH is about 4-5.

Human type I collagen, the major collagen in bone is a good substrate for cathepsin K. See Kafienah, W., et al., 1998, Biochem J 331:727-732, which is hereby incorporated by reference in its entirety. In vitro experiments using antisense oligonucleotides to cathepsin K, have shown diminished bone resorption in vitro, which is probably due to a reduction in translation of cathepsin K mRNA. See Inui, T., et al., 1997, J Biol Chem 272:8109-8112, which is hereby incorporated by reference in its entirety. The crystal structure of cathepsin K has been resolved. See McGrath, M. E., et al., 1997, Nat Struct Biol 4:105-109; Zhao, B., et al., 1997, Nat Struct Biol 4: 109-11, which are hereby incorporated by reference in their entirety. Also, selective peptide based inhibitors of cathepsin K have been developed See Bromme, D., et al., 1996, Biochem J 315:85-89; Thompson, S. K., et al., 1997, Proc Natl Acad Sci USA 94:14249-14254, which are hereby incorporated by reference in their entirety. Accordingly, inhibitors of Cathepsin K can reduce bone resorption. Such inhibitors would be useful in treating disorders involving bone resorption, such as osteoporosis.

### SUMMARY OF THE INVENTION

The present invention relates to compounds that are capable of treating and/or preventing cathepsin dependent conditions or disease states in a mammal in need thereof. One embodiment of the present invention is illustrated by a compound of Formula I, and the pharmaceutically acceptable salts, stereoisomers and N-oxide derivatives thereof:

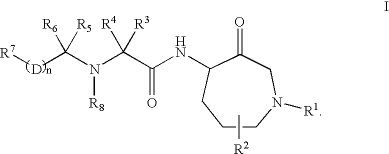

### DETAILED DESCRIPTION OF THE INVENTION

The present invention relates to compounds of the following chemical formula: 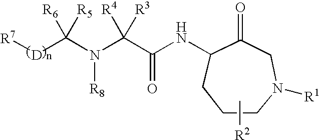
wherein R¹ is hydrogen, C₁₋₆ alkyl, —SO₂R⁹, —C(O)R⁹ or arylC₁₋₆alkyl;
R² is hydrogen, C₁₋₆ alkyl or C₃₋₆ cycloalkyl;
R³ is hydrogen, C₁₋₆ alkyl or C₂₋₆ alkenyl wherein said alkyl and alkenyl groups are optionally substituted with C₃₋₆ cycloalkyl or halo;
R⁴ is hydrogen, C₁₋₆ alkyl or C₂₋₆ alkenyl wherein said alkyl and alkenyl groups are optionally substituted with C₃₋₆ cycloalkyl or halo;
or R³ and R⁴ can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted with C₁₋₆ alkyl, halo, hydroxyalkyl, hydroxy, alkoxy or keto;
R⁵ is selected from hydrogen or C₁₋₆ alkyl substituted with 1-6 halo;
R⁶ is aryl, heteroaryl, C₁₋₆ haloalkyl, arylalkyl or heteroarylalkyl, wherein said aryl, heteroaryl, arylalkyl and heteroarylalkyl groups are optionally substituted with halo, C₁₋₆ alkyl, C₁₋₆ haloalkyl, C₃₋₆ cycloalkyl, —SR⁹, —SR¹², —SOR⁹, —SOR¹², —SO₂R⁹, —SO₂R^{12,} —SO₂CH(R¹²)(R¹¹), —OR¹², —N(R¹⁰)(R¹¹) or cyano;
D is C₁₋₃ alkyl, C₂₋₃ alkenyl, C₂₋₃ alkynyl, aryl, heteroaryl, C₃₋₈ cycloalkyl or heterocyclyl wherein said aryl, heteroaryl, cycloalkyl and heterocyclyl groups, which may be monocyclic or bicyclic, are optionally substituted on either the carbon or the heteroatom with one to five substituents selected from C₁₋₆ alkyl, halo or keto;
R⁷ is hydrogen, C₁₋₆ alkyl, C₂₋₆ alkenyl, C₂₋₆ alkynyl, C₁₋₆ alkyloxy, halo, nitro, cyano, aryl, heteroaryl, C₃₋₈ cycloalkyl, heterocyclyl, —C(O)OR¹⁰, —C(O)OSi[CH(CH₃)₂]₃, —OR¹⁰, —C(O)R¹⁰, —R¹⁰C(O)R⁹, —C(O)R⁹, —C(O)N(R¹²)(R¹²), —C(O)N(R¹⁰)(R¹¹), —C(R¹⁰)(R¹¹)OH, —SR¹², —SR⁹, —R¹⁰SR⁹, —R⁹, —C(R⁹)₃, —C(R¹⁰)(R¹¹)N(R⁹)₂, —NR¹⁰C(O)NR¹⁰S(O)₂R⁹, —SO₂R¹², —SO(R¹²), —SO₂R⁹, —SO₂N(R^{c})(R^{d}), —SO₂CH(R¹⁰)(R¹¹), —SO₂N(R¹⁰)C(O)(R¹²), —SO₂(R¹⁰)C(O)N(R¹²)₂, —OSO₂R¹⁰, —N(R¹⁰)(R¹¹), —N(R¹⁰)C(O)N(R¹⁰)(R⁹), —N(R¹⁰)C(O)R¹⁰, —N(R¹⁰)C(O)OR¹⁰, —N(R¹⁰)SO₂(R¹⁰), —C(R¹⁰)(R¹¹)NR¹⁰C(R¹⁰)(R¹¹)R⁹, —C(R¹⁰)(R¹¹)N(R¹⁰)R⁹, —C(R¹⁰)(R¹¹)N(R¹⁰)(R¹¹), —C(R¹⁰)(R¹¹)SC(R¹⁰)(R¹¹)R⁹, R¹⁰S—, —C(R^{a})(R^{b})NR^{a}C(R^{a})(R^{b})₂, —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁹, —C(O)C(R^{a})(R^{b})S(R^{a})(R^{b}) or C(R^{a})(R^{b})C(O)N(R^{a})(R^{b}); wherein said groups are optionally substituted on either the carbon or the heteroatom with one to five substituents independently selected from C₁₋₆ alkyl, halo, keto, cyano, haloalkyl, hydroxyalkyl, —OR⁹, —O(aryl), —NO₂, —NH₂, —NHS(O)₂R⁸, —R⁹SO₂R¹², SO₂R¹², SO(R¹²), SO₂N(R^{c})(R^{d}), SO₂N(R¹⁰)C(O)(R¹²), —C(R¹⁰)(R¹¹)N(R¹⁰)(R¹¹), —C(R¹⁰)(R¹¹)OH, —COOH, —C(R^{a})(R^{b})C(O)N(R^{a})(R^{b}), —N(R¹⁰)C(R¹⁰)(R¹¹), —NH(CH₂)₂OH, —NHC(O)OR¹⁰, Si(CH₃)₃, heterocycyl, aryl or heteroaryl;
R⁸ is hydrogen or C₁₋₆ alkyl;
or R⁴ and R⁸ or can be taken together with any of the atoms to which they may be attached or are between them to form a 4-10 membered heterocyclyl ring system wherein said ring system, which may be monocyclic or bicyclic, is optionally substituted with C₁₋₆ alkyl, halo, hydroxyalkyl, hydroxy, keto, OR¹⁰, SR¹⁰ or N(R¹⁰)₂;
R⁹ is selected from the group consisting of hydrogen, aryl, aryl(C₁₋₄) alkyl, heteroaryl, heteroaryl(C₁₋₄)alkyl, C₃₋₈cycloalkyl, C₃₋₈cycloalkyl(C₁₋₄)alkyl, and heterocyclyl(C₁₋₄)alkyl wherein said groups can be optionally substituted with halo or alkoxy;
R¹⁰ is hydrogen or C₁₋₆ alkyl;
R¹¹ is hydrogen or C₁₋₆ alkyl;
R¹² is hydrogen or C₁₋₆ alkyl which is optionally substituted with halo, alkoxy, cyano, —NR¹⁰ or —SR¹⁰;
R^{a} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with C₁₋₆ alkyl or halo;
R^{b} is hydrogen, C₁₋₆ alkyl, (C₁₋₆ alkyl)aryl, (C₁₋₆alkyl)hydroxyl, alkoxyl, hydroxyl, halo, aryl, heteroaryl, C₃₋₈ cycloalkyl, heterocyclyl,wherein said alkyl, aryl, heteroaryl, C₃₋₈ cycloalkyl and heterocyclyl can be optionally substituted on either the carbon or the heteroatom with C₁₋₆ alkyl or halo;
or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with C₁₋₆ alkyl and halo;
R^{c} is hydrogen or C₁₋₆ alkyl which is optionally substituted with halo or OR⁹;
R^{d} is hydrogen or C₁₋₆ alkyl which is optionally substituted with halo or OR⁹;
or R^{c} and R^{d} can be taken together with the nitrogen atom to which they are attached or are between them to form a C₃₋₈ heterocyclyl ring which is optionally substituted with C₁₋₆ alkyl, halo hydroxyalkyl, hydroxy, alkoxy or keto;
n is independently selected from an integer from zero to three;
and the pharmaceutically acceptable salts, stereoisomers and N-oxide derivatives thereof.

In an embodiment of the invention, R¹ is —SO₂R⁹and R² is hydrogen. In an embodiment of the invention, R³ and R⁴ are each independently C₁₋₄ alkyl or H. In a further embodiment of the invention R³ is isobutyl and R⁴ is H. In another embodiment of the invention, R³ and R⁴, when on the same carbon atom, can be taken together with the carbon atom to which they are attached to form C₃₋₈ cycloalkyl ring, C₅₋₈ cycloalkenyl ring, or five to seven membered heterocyclyl wherein said cycloalkyl, cycloalkenyl and heterocyclyl groups are optionally substituted with C₁₋₆ alkyl, halo, hydroxyalkyl, hydroxy, alkoxy or keto. Examples of ring systems that can be formed include, but are not limited to the following, keeping in mind that the heterocycle is optionally substituted with one or more substituents as described above: cyclopropyl, cyclobutyl, cyclopentyl, cyclohexyl and cycloheptyl. A preferred embodiment is when cyclohexyl is formed.

In an embodiment of the invention, R⁵ is C₁₋₆ alkyl substituted with 1-6 halo and R⁶ is C₁₋₆ alkyl substituted with 1-6 halo. In another embodiment of the invention, R⁵ is hydrogen and R⁶ is C₁₋₆ alkyl substituted with 1-6 halo. In a further embodiment, R⁵ is hydrogen and R⁶ is C₁₋₆ alkyl substituted with 1-6 fluoro. In a further embodiment, R⁵ is hydrogen and R⁶ is C₁₋₃ alkyl substituted with 3 fluoro. In another embodiment of the invention, R⁵ is hydrogen and R⁶ is aryl or heteroaryl, wherein said aryl and heteroaryl are optionally substituted with halo or —SO₂R¹².

In an embodiment of the invention, R⁴ and R⁸ or can be taken together with any of the atoms to which they may be attached or are between them to form a 4-10 membered heterocyclyl ring system wherein said ring system, which may be monocyclic or bicyclic, is optionally substituted with C₁₋₆ alkyl, halo, hydroxyalkyl, hydroxy, keto, —OR¹⁰, —SR¹⁰ or —N(R¹⁰)₂. In a further embodiment of the invention, R⁴ and R⁸ are defined such that they can be taken together with the nitrogen to which they are attached to form a monocyclic or bicyclic heterocyclyl with 5-7 members in each ring and optionally containing, in addition to the nitrogen, 1 or 2 additional heteroatoms selected from N, O and S, said heterocycle optionally substituted with one or more substituents selected from C₁₋₆ alkyl, halo, hydroxyalkyl, hydroxy, keto, —OR¹⁰, —SR¹⁰ or —N(R¹⁰)₂. Nonlimiting examples of heterocyclyl ring systems that can be formed include piperazinyl, piperidinyl, pyrrolidinyl and the like. In a further example, R⁴ and R⁸ are defined such that they can be taken together with the nitrogen to which they are attached to form a 5 or 6 membered heterocyclyl ring system. Examples of the heterocycles that can thus be formed include, but are not limited five or six membered rings containing at least one nitrogen, which is optionally substituted with one or more substituents as described above. A preferred embodiment is when optionally substituted pyrolidinyl is formed.

In another embodiment of the invention, R^{a} and R^{b}, can be taken together with the carbon atom to which they are attached or are between them to form a C₃₋₈ cycloalkyl ring or a C₃₋₈ heterocyclyl ring wherein the cycloalkyl and heterocyclyl systems are optionally substituted with C₁₋₆ alkyl and halo. Examples of ring systems that can be formed include, but are not limited to the following, cyclopropyl, cyclobutyl, cyclopentyl, cyclohexyl, cycloheptyl, imidazolyl, piperazinyl, piperidinyl, pyrrolidinyl and the like.

Embodied by the present invention are methods for treating disorders related to abnormal bone resoprtion. Such disorders include, but are not limited to, osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, metastatic bone disease, hypercalcemia of malignancy, and multiple myeloma. A preferred embodiment includes methods for treating osteoporosis and metastatic bone disease. A more preferred embodiment includes methods for treating osteoporosis.

Specific embodiments of the present invention include, but are not limited to: N¹-[3-Oxo-1-(pyridin-2-ylsulfonyl)azepan-4-yl]-N²-{(1S)-2,2,2-trifluoro-1-[4′-(methylsulfonyl)-1,1′-biphenyl-4-yl]ethyl}-L-leucinamide, and the pharmaceutically acceptable salts, stereoisomers and N-oxide derivatives thereof.

Also included within the scope of the present invention is a pharmaceutical composition which is comprised of a compound of Formula I as described above and a pharmaceutically acceptable carrier. The invention is also contemplated to encompass a pharmaceutical composition which is comprised of a pharmaceutically acceptable carrier and any of the compounds specifically disclosed in the present application. These and other aspects of the invention will be apparent from the teachings contained herein.

Utilities

The compounds of the present invention are inhibitors of cathepsins and are therefore useful to treat or prevent cathepsin dependent diseases or conditions in mammals, preferably humans. Specifically, the compounds of the present invention are inhibitors of Cathepsin K and are therefore useful to treat or prevent Cathepsin K dependent diseases or conditions in mammals, preferably humans.

“Cathepsin dependent diseases or conditions” refers to pathologic conditions that depend on the activity of one or more cathepsins. “Cathepsin K dependent diseases or conditions” refers to pathologic conditions that depend on the activity of Cathepsin K. Diseases associated with Cathepsin K activities include osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, metastatic bone disease, hypercalcemia of malignancy, and multiple myeloma. In treating such conditions with the instantly claimed compounds, the required therapeutic amount will vary according to the specific disease and is readily ascertainable by those skilled in the art. Although both treatment and prevention are contemplated by the scope of the invention, the treatment of these conditions is the preferred use.

An embodiment of the invention is a method of inhibiting cathepsin activity in a mammal in need thereof, comprising administering to the mammal a therapeutically effective amount of any of the compounds or any of the pharmaceutical compositions described above.

A class of the embodiment is the method wherein the cathepsin activity is cathepsin K activity.

Another embodiment of the invention is a method of treating or preventing cathepsin dependent conditions in a mammal in need thereof, comprising administering to the mammal a therapeutically effective amount of any of the compounds or any of the pharmaceutical compositions described above.

A class of the embodiment is the method wherein the cathepsin activity is cathepsin K activity.

Another embodiment of the invention is a method of inhibiting bone loss in a mammal in need thereof, comprising administering to the mammal a therapeutically effective amount of any of the compounds or any of the pharmaceutical compositions described above. Another embodiment of the invention is a method of reducing bone loss in a mammal in need thereof, comprising administering to the mammal a therapeutically effective amount of any of the compounds or any of the pharmaceutical compositions described above. The utility of cathepsin K inhibitors in the inhibition of bone resorption is known in the literature, see Stroup, G. B., Lark, M. W., Veber, D F., Bhattacharrya, A., Blake, S., Dare, L. C., Erhard, K. F., Hoffman, S. J., James, I. E., Marquis, R. w., Ru, Y., Vasko-Moser, J. A., Smith, B. R., Tomaszek, T. and Gowen, M. Potent and selective inhibition of human cathepsin K leads to inhibition of bone resorption in vivo in a nonhuman primate. J. Bone Miner. Res., 16:1739-1746;2001; and Votta, B. J., Levy, M. A., Badger, A., Dodds, R. A., James, I. E., Thompson, S., Bossard, M. J., Carr, T., Connor, J. R., Tomaszek, T. A., Szewczuk, L., Drake, F. H., Veber, D., and Gowen, M. Peptide aldehyde inhibitors of cathepsin K inhibit bone resorption both in vivo and in vitro. J. Bone Miner. Res. 12:1396-1406; 1997.

Another embodiment of the invention is a method of treating or preventing osteoporosis in a mammal in need thereof, comprising administering to the mammal a therapeutically effective amount of any of the compounds or any of the above pharmaceutical compositions described above. The utility of cathepsin K inhibitors in the treatment or prevention of osteoporosis is known in the literature, see Saftig, P., Hunziker, E., Wehmeyer, O., Jones, S., Boyde, A., Rommerskirch, W., Moritz, J. D., Schu, P., and Vonfigura, K. Impaired osteoclast bone resorption leads to osteoporosis in cathepsin K-deficient mice. Proc. Natl. acad. Sci. USA 95:13453-13458; 1998.

Another embodiment of the invention is a method treating cancer in a mammal in need thereof, comprising administering to the mammal a therapeutically effective amount of any of the compounds or any of the pharmaceutical compositions described above. It is known in the literature that Cathepsin K is expressed in human breast carcinoma, see Littlewood-Evans A J, Bilbe G, Bowler W B, Farley D, Wlodarski B, Kokubo T, Inaoka T, Sloane J, Evans D B, Gallagher J A, “The osteoclast-associated protease cathepsin K is expressed in human breast carcinoma.”Cancer Res Dec. 1, 1997; 57(23):5386-90.

Exemplifying the invention is the use of any of the compounds described above in the preparation of a medicament for the treatment and/or prevention of osteoporosis in a mammal in need thereof. Still further exemplifying the invention is the use of any of the compounds described above in the preparation of a medicament for the treatment and/or prevention of: bone loss, bone resorption, bone fractures, metastatic bone disease and/or disorders related to cathepsin functioning.

The compounds of this invention may be administered to mammals, preferably humans, either alone or, preferably, in combination with pharmaceutically acceptable carriers or diluents, optionally with known adjuvants, such as alum, in a pharmaceutical composition, according to standard pharmaceutical practice. The compounds can be administered orally or parenterally, including the intravenous, intramuscular, intraperitoneal, subcutaneous, rectal and topical routes of administration.

In the case of tablets for oral use, carriers which are commonly used include lactose and corn starch, and lubricating agents, such as magnesium stearate, are commonly added. For oral administration in capsule form, useful diluents include lactose and dried corn starch. For oral use of a therapeutic compound according to this invention, the selected compound may be administered, for example, in the form of tablets or capsules, or as an aqueous solution or suspension. For oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polyactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compounds of the present invention can be used in combination with other agents useful for treating cathepsin-mediated conditions. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating cathepsin-mediated conditions includes in principle any combination with any pharmaceutical composition useful for treating disorders related to estrogen functioning.

The instant compounds are also useful in combination with known agents useful for treating or preventing osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, metastatic bone disease, hypercalcemia of malignancy, and multiple myeloma. Combinations of the presently disclosed compounds with other agents useful in treating or preventing osteoporosis or other bone disorders are within the scope of the invention. A person of ordinary skill in the art would be able to discern which combinations of agents would be useful based on the particular characteristics of the drugs and the disease involved. Such agents include the following: an organic bisphosphonate; an estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; and the pharmaceutically acceptable salts and mixtures thereof. A preferred combination is a compound of the present invention and an organic bisphosphonate. Another preferred combination is a compound of the present invention and an estrogen receptor modulator. Another preferred combination is a compound of the present invention and an androgen receptor modulator. Another preferred combination is a compound of the present invention and an osteoblast anabolic agent.

“Organic bisphosphonate” includes, but is not limited to, compounds of the chemical formula 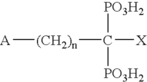
wherein n is an integer from 0 to 7 and wherein A and X are independently selected from the group consisting of H, OH, halogen, NH₂, SH, phenyl, C1-C30 alkyl, C3-C30 branched or cycloalkyl, bicyclic ring structure containing two or three N, C1-C30 substituted alkyl, C1-C10 alkyl substituted NH₂, C3-C10 branched or cycloalkyl substituted NH₂, C1-C10 dialkyl substituted NH₂, C1-C10 alkoxy, C1-C10 alkyl substituted thio, thiophenyl, halophenylthio, C1-C10 alkyl substituted phenyl, pyridyl, furanyl, pyrrolidinyl, imidazolyl, imidazopyridinyl, and benzyl, such that both A and X are not selected from H or OH when n is 0; or A and X are taken together with the carbon atom or atoms to which they are attached to form a C3-C10 ring.

In the foregoing chemical formula, the alkyl groups can be straight, branched, or cyclic, provided sufficient atoms are selected for the chemical formula. The C1-C30 substituted alkyl can include a wide variety of substituents, nonlimiting examples which include those selected from the group consisting of phenyl, pyridyl, furanyl, pyrrolidinyl, imidazonyl, NH₂, C1-C10 alkyl or dialkyl substituted NH₂, OH, SH, and C1-C10 alkoxy.

The foregoing chemical formula is also intended to encompass complex carbocyclic, aromatic and hetero atom structures for the A and/or X substituents, nonlimiting examples of which include naphthyl, quinolyl, isoquinolyl, adamantyl, and chlorophenylthio.

Pharmaceutically acceptable salts and derivatives of the bisphosphonates are also useful herein. Non-limiting examples of salts include those selected from the group consisting alkali metal, alkaline metal, ammonium, and mono-, di-, tri-, or tetra-C1-C30-alkyl-substituted ammonium. Preferred salts are those selected from the group consisting of sodium, potassium, calcium, magnesium, and ammonium salts. More preferred are sodium salts. Non-limiting examples of derivatives include those selected from the group consisting of esters, hydrates, and amides.

It should be noted that the terms “bisphosphonate” and “bisphosphonates”, as used herein in referring to the therapeutic agents of the present invention are meant to also encompass diphosphonates, biphosphonic acids, and diphosphonic acids, as well as salts and derivatives of these materials. The use of a specific nomenclature in referring to the bisphosphonate or bisphosphonates is not meant to limit the scope of the present invention, unless specifically indicated. Because of the mixed nomenclature currently in use by those of ordinary skill in the art, reference to a specific weight or percentage of a bisphosphonate compound in the present invention is on an acid active weight basis, unless indicated otherwise herein. For example, the phrase “about 5 mg of a bone resorption inhibiting bisphosphonate selected from the group consisting of alendronate, pharmaceutically acceptable salts thereof, and mixtures thereof, on an alendronic acid active weight basis” means that the amount of the bisphosphonate compound selected is calculated based on 5 mg of alendronic acid.

Non-limiting examples of bisphosphonates useful herein include the following:

Alendronate, also known as Alendronic acid, 4-amino-1-hydroxybutylidene-1,1-bisphosphonic acid, alendronate sodium, alendronate monosodium trihydrate or 4-amino-1-hydroxybutylidene-1,1-bisphosphonic acid monosodium trihydrate

Alendronate is described in U.S. Pat. No. 4,922,007, to Kieczykowski et al., issued May 1, 1990; U.S. Pat. No. 5,019,651, to Kieczykowski et al., issued May 28, 1991; U.S. Pat. No. 5,510,517, to Dauer et al., issued Apr. 23, 1996; U.S. Pat. No. 5,648,491, to Dauer et al., issued Jul. 15, 1997, all of which are incorporated by reference herein in their entirety.

Cycloheptylaminomethylene-1,1-bisphosphonic acid, YM 175, Yamanouchi (incadronate, formerly known as cimadronate), as described in U.S. Pat. No. 4,970,335, to Isomura et al., issued Nov. 13, 1990, which is incorporated by reference herein in its entirety.

1,1-dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety.

1-hydroxy-3-(1-pyrrolidinyl)-propylidene-1,1-bisphosphonic acid (EB-1053).

1-hydroxyethane-1,1-diphosphonic acid (etidronic acid).

1-hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety.

1-hydroxy-2-imidazo-(1,2-a)pyridin-3-yethylidene (minodronate).

6-amino-1-hydroxyhexylidene-1,1-bisphosphonic acid (neridronate).

3-(dimethylamino)-1-hydroxypropylidene-1,1-bisphosphonic acid (olpadronate).

3-amino-1-hydroxypropylidene-1,1-bisphosphonic acid (pamidronate).

[2-(2-pyridinyl)ethylidene]-1,1-bisphosphonic acid (piridronate) is described in U.S. Pat. No. 4,761,406, which is incorporated by reference in its entirety.

1-hydroxy-2-(3-pyridinyl)-ethylidene-1,1-bisphosphonic acid (risedronate), is described in U.S. Pat. No. 5,583,122, which is incorporated by reference in its entirety.

(4-chlorophenyl)thiomethane-1,1-disphosphonic acid (tiludronate) as described in U.S. Pat. No. 4,876,248, to Breliere et al., Oct. 24, 1989, which is incorporated by reference herein in its entirety.

1-hydroxy-2-(1H-imidazol-1-yl)ethylidene-1,1-bisphosphonic acid (zoledronate).

Nonlimiting examples of bisphosphonates include alendronate, cimadronate, clodronate, etidronate, ibandronate, incadronate, minodronate, neridronate, olpadronate, pamidronate, piridronate, risedronate, tiludronate, and zolendronate, and pharmaceutically acceptable salts and esters thereof. A particularly preferred bisphosphonate is alendronate, especially a sodium, potassium, calcium, magnesium or ammonium salt of alendronic acid. Exemplifying the preferred bisphosphonate is the sodium salt of alendronate, especially a hydrated sodium salt of alendronate. The salt can be hydrated with a whole number of moles of water or non whole numbers of moles of water. Further exemplifying the preferred bisphosphonate is a hydrated sodium salt of alendronate, especially when the hydrated salt is alendronate monosodium trihydrate.

It is recognized that mixtures of two or more of the bisphosphonate actives can be utilized.

The precise dosage of the organic bisphosphonate will vary with the dosing schedule, the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. Appropriate amounts can be determined by routine experimentation from animal models and human clinical studies. Generally, an appropriate amount of bisphosphonate is chosen to obtain a bone resorption inhibiting effect, i.e. a bone resorption inhibiting amount of the bisphosphonate is administered. For humans, an effective oral dose of bisphosphonate is typically from about 1.5 to about 6000 μg/kg body weight and preferably about 10 to about 2000 μg/kg of body weight. For alendronate monosodium trihydrate, common human doses which are administered are generally in the range of about 2 mg/day to about 40 mg/day, preferably about 5 mg/day to about 40 mg/day. In the U.S. presently approved dosages for alendronate monosodium trihydrate are 5 mg/day for preventing osteoporosis, 10 mg/day for treating osteoporosis, and 40 mg/day for treating Paget's disease.

In alternative dosing regimens, the bisphosphonate can be administered at intervals other than daily, for example once-weekly dosing, twice-weekly dosing, biweekly dosing, and twice-monthly dosing. In a once weekly dosing regimen, alendronate monosodium trihydrate would be administered at dosages of 35 mg/week or 70 mg/week.

“Estrogen receptor modulators” refers to compounds which interfere or inhibit the binding of estrogen to the receptor, regardless of mechanism. Examples of estrogen receptor modulators include, but are not limited to, estrogen, progestogen, estradiol, droloxifene, raloxifene, lasofoxifene, TSE-424, tamoxifen, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

Non-steroidal compounds having androgen receptor modulating properties are disclosed in U.S. Pat. Nos. 5,688,808; 5,696,130; 6,017,924; 6,093,821; WO 01/16139 (published 8 Mar. 2001); and WO 01/16108 (published 8 Mar. 2001), all assigned to ligand Pharmaceuticals, and in WO 01/27086, assigned to Kaken Pharm. Co. Additional background for the rationale behind the development of Selective Androgen Receptor Modulators is found in L. Zhi and E. Martinborough in Ann. Rep. Med. Chem. 36: 169-180 (2001). Non-steroidal SARMs were disclosed in J. P. Edwards, “New Nonsteroidal Androgen Receptor Modulators Based on 4-(Trifluoromethyl)-2(1H)-Pyrrolidino[3,2-g]quinolinone,” Bioorg. Med. Chem. Lett., 8: 745-750 (1998) and in L. Zhi et al., “Switching Androgen Receptor Antagonists to Agonists by Modifying C-ring Substituents on Piperidino[3,4-g]quinolinone,” Biorg. Med. Chem. Lett., 9: 1009-1012 (1999).

“An inhibitor of osteoclast proton ATPase” refers to an inhibitor of the proton ATPase, which is found on the apical membrane of the osteoclast, and has been reported to play a significant role in the bone resorption process. This proton pump represents an attractive target for the design of inhibitors of bone resorption which are potentially useful for the treatment and prevention of osteoporosis and related metabolic diseases. See C. Farina et al., “Selective inhibitors of the osteoclast vacuolar proton ATPase as novel bone antiresorptive agents,” DDT, 4: 163-172 (1999)), which is hereby incorporated by reference in its entirety.

“HMG-CoA reductase inhibitors” refers to inhibitors of 3-hydroxy-3-methylglutaryl-CoA reductase. Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33. The terms “HMG-CoA reductase inhibitor” and “inhibitor of HMG-CoA reductase” have the same meaning when used herein.

Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention. An illustration of the lactone portion and its corresponding open-acid form is shown below as structures I and II.

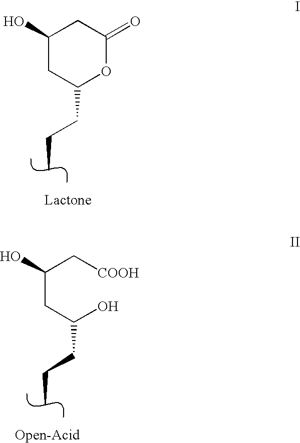

In HMG-CoA reductase inhibitors where an open-acid form can exist, salt and ester forms may preferably be formed from the open-acid, and all such forms are included within the meaning of the term “HMG-CoA reductase inhibitor” as used herein. Preferably, the HMG-CoA reductase inhibitor is selected from lovastatin and simvastatin, and most preferably simvastatin. Herein, the term “pharmaceutically acceptable salts” with respect to the HMG-CoA reductase inhibitor shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenz-imidazole, diethylamine, piperazine, and tris(hydroxymethyl)aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Ester derivatives of the described HMG-CoA reductase inhibitor compounds may act as prodrugs which, when absorbed into the bloodstream of a warm-blooded animal, may cleave in such a manner as to release the drug form and permit the drug to afford improved therapeutic efficacy.

As used above, “integrin receptor antagonists” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins. H.N. Lode and coworkers in PNAS USA 96: 1591-1596 (1999) have observed synergistic effects between an antiangiogenic αv integrin antagonist and a tumor-specific antibody-cytokine (interleukin-2) fusion protein in the eradication of spontaneous tumor metastases. Their results suggested this combination as having potential for the treatment of cancer and metastatic tumor growth. αᵥβ₃ integrin receptor antagonists inhibit bone resorption through a new mechanism distinct from that of all currently available drugs. Integrins are heterodimeric transmembrane adhesion receptors that mediate cell-cell and cell-matrix interactions. The α and β integrin subunits interact non-covalently and bind extracellular matrix ligands in a divalent cation-dependent manner. The most abundant integrin on osteoclasts is αᵥβ₃ (>10⁷/osteoclast), which appears to play a rate-limiting role in cytoskeletal organization important for cell migration and polarization: The αᵥβ₃ antagonizing effect is selected from inhibition of bone resorption, inhibition of restenosis, inhibition of macular degeneration, inhibition of arthritis, and inhibition of cancer and metastatic growth. Nonlimiting examples of integrin receptor antagonists, and methods for their preparation, are found in U.S. Pat. No. 5,925,655 (issued Jul. 20, 1999), U.S. Pat. No. 6,211,184 (issued Apr. 3, 2001), U.S. Pat. No. 5,919,792 (issued Jul. 6, 1999), U.S. Pat. No. 5,952,792 (issued Sep. 14, 1999), U.S. Pat. No. 6,017,925 (issued Jan. 25, 2000), U.S. Pat. No. 6,048,861 (issued Apr. 11, 2000), U.S. Pat. No. 6,232,308 (issued May 15, 2001), U.S. Pat. No. 6,358,970 (issued Mar. 19, 2002), U.S. Pat. No. 6,040,311 (issued Mar. 21, 2000), U.S. Pat. No. 6,066,648 (issued May 23, 2000), U.S. Pat. No. 6,211,191 (issued Apr. 3, 2001), U.S. Pat. No. 6,017,926 (issued Jan. 25, 2000), U.S. Pat. No. 6,090,944 (Jul. 18, 2000), U.S. Pat. No. 6,410,526 (issued Jun. 25, 2002), U.S. Pat. No. 6,413,955 (issued Jul. 2, 2002), U.S. Pat. No. 6,426,353 (issued Jul. 30, 2002), U.S. Pat. No. 6,444,680 (issued Sep. 3, 2002), and in PCT International Publication Numbers WO 00/48603 (published Aug. 24, 2000), WO 01/53297 (published Jul. 26, 2001), WO 01/53262 (published Jul. 26, 2001), WO 02/22616 (published Mar. 21, 2002), WO 02/07730 (published Jan. 31, 2002), WO 02/28840 (published Apr. 11, 2002), WO 02/40505 (published May 23, 2002).

“An osteoblast anabolic agent” refers to agents that build bone, such as PTH. The intermittent administration of parathyroid hormone (PTH) or its amino-terminal fragments and analogues have been shown to prevent, arrest, partially reverse bone loss and stimulate bone formation in animals and humans. For a discussion refer to D. W. Dempster et al., “Anabolic actions of parathyroid hormone on bone,” Endocr Rev 14: 690-709 (1993). Studies have demonstrated the clinical benefits of parathyroid hormone in stimulating bone formation and thereby increasing bone mass and strength. Results were reported by R M Neer et al., in New Eng J Med 344 1434-1441 (2001).

In addition, parathyroid hormone-related protein fragments or analogues, such as PTHrP-(1-36) have demonstrated potent anticalciuric effects [see M. A. Syed et al., “Parathyroid hormone-related protein-(1-36) stimulates renal tubular calcium reabsorption in normal human volunteers: implications for the pathogenesis of humoral hypercalcemia of malignancy,” JCEM 86: 1525-1531 (2001)) and may also have potential as anabolic agents for treating osteoporosis.

If formulated as a fixed dose, such combination products employ the compounds of this invention within the dosage range described below and the other pharmaceutically active agent(s) within its approved dosage range. Compounds of the instant invention may alternatively be used sequentially with known pharmaceutically acceptable agent(s) when a combination formulation is inappropriate.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents. The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The terms “treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

The term “bone resorption,” as used herein, refers to the process by which osteoclasts degrade bone.

The present invention also encompasses a pharmaceutical composition useful in the treatment of osteoporosis or other bone disorders, comprising the administration of a therapeutically effective amount of the compounds of this invention, with or without pharmaceutically acceptable carriers or diluents. Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

When a compound according to this invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for a cathepsin dependent condition. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittant throughout the dosage regimen.

These and other aspects of the invention will be apparent from the teachings contained herein.

Definitions

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

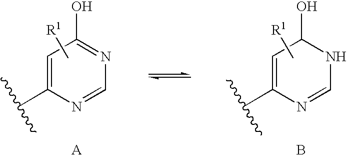

When any variable (e.g. R¹, R², R^{a} etc.) occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring carbon atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear, branched, or cyclic arrangement. For example, “C₁-C₁₀ alkyl” specifically includes methyl, ethyl, propyl, butyl, pentyl, hexyl, heptyl, octyl, nonyl, decyl, and so on. “Alkoxy” represents an alkyl group of indicated number of carbon atoms attached through an oxygen bridge.

The term “cycloalkyl” or “carbocycle” shall mean cyclic rings of alkanes of three to eight total carbon atoms, or any number within this range (i.e., cyclopropyl, cyclobutyl, cyclopentyl, cyclohexyl, cycloheptyl or cyclooctyl).

If no number of carbon atoms is specified, the term “alkenyl” refers to a non-aromatic hydrocarbon radical, straight or branched, containing from 2 to 10 carbon atoms and at least 1 carbon to carbon double bond. Preferably 1 carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “cycloalkenyl” shall mean cyclic rings of 3 to 10 carbon atoms and at least 1 carbon to carbon double bond (i.e., cycloprenpyl, cyclobutenyl, cyclopenentyl, cyclohexenyl, cycloheptenyl or cycloocentyl).

The term “alkynyl” refers to a hydrocarbon radical straight or branched, containing from 2 to 10 carbon atoms and at least 1 carbon to carbon triple bond. Up to 3 carbon-carbon triple bonds may be present. Thus, “C₂-C₆ alkynyl” means an alkynyl radical having from 2 to 6 carbon atoms. Alkynyl groups include ethynyl, propynyl and butynyl. As described above with respect to alkyl, the straight, branched or cyclic portion of the alkynyl group may contain triple bonds and may be substituted if a substituted alkyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or tricyclic ring of up to 10 atoms in each ring, wherein-at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo” or “halogen” as used herein is intended to include chloro, fluoro, bromo and iodo. The term “keto” means carbonyl (C═O). The term “alkoxy” as used herein means an alkyl portion, where alkyl is as defined above, connected to the remainder of the molecule via an oxygen atom. Examples of alkoxy include methoxy, ethoxy and the like.

The term “haloalkyl” includes an alkyl portion, where alkyl is as defined above, which is substituted with one to five halo.

The term “arylalkyl” includes an alkyl portion where alkyl is as defined above and to include an aryl portion where aryl is as defined above. Examples of arylalkyl include, but are not limited to, benzyl, fluorobenzyl, chlorobenzyl, phenylethyl, phenylpropyl, fluorophenylethyl, and chlorophenylethyl. Examples of alkylaryl include, but are not limited to, toluyl, ethylphenyl, and propylphenyl.

The term “heteroarylalkyl” as used herein, shall refer to a system that includes a heteroaryl portion, where heteroaryl is as defined above, and contains an alkyl portion. Examples of heteroarylalkyl include, but are not limited to, thienylmethyl, thienylethyl, thienylpropyl, pyridylmethyl, pyridylethyl and imidazoylmethyl

The term “hydroxyalkyl” means a linear monovalent hydrocarbon raidcal of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, and the like.

The term “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also emcompassed by this definition.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. Also when compounds of Formula I contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. 1981, the disclosure of which is incorporated herein by reference in its entirety. The protected derivatives of compounds of Formula I can be prepared by methods well known in the art.

The alkyl, alkenyl, alkynyl, cycloalkyl, aryl, heteroaryl and heterocyclyl substituents may be unsubstituted or unsubstituted, unless specifically defined otherwise. For example, a (C₁-C₆)alkyl may be substituted with one or more substituents selected from OH, oxo, halogen, alkoxy, dialkylamino, or heterocyclyl, such as morpholinyl, piperidinyl, and so on. In the case of a disubstituted alkyl, for instance, wherein the substituents are oxo and OH, the following are included in the definition: —(C═O)CH₂CH(OH)CH₃, —(C═O)OH, —CH₂(OH)CH₂CH(O), and so on.

Whenever the term “alkyl” or “aryl” or either of their prefix roots appear in a name of a substituent (e.g., aryl C₀₋₈ alkyl) it shall be interpreted as including those limitations given above for “alkyl” and “aryl.” Designated numbers of carbon atoms (e.g., C₁₋₁₀) shall refer independently to the number of carbon atoms in an alkyl or cyclic alkyl moiety or to the alkyl portion of a larger substituent in which alkyl appears as its prefix root.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The preparation of the pharmaceutically acceptable salts described above and other typical pharmaceutically acceptable salts is more fully described by Berg et al., “Pharmaceutical Salts,” J. Pharm. Sci., 1977:66:1-19, hereby incorporated by reference. The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

For purposes of this specification, the following abbreviations have the indicated meanings:
BuLi=butyl lithium
Bu₄NHSO₄=butyl aminosulfate
CH₂Cl₂=methylene chloride
CrO₃=chromium oxide
DMAP=4-(dimethylamino)pyridine
Et₃N=triethylamine
EtOAc=ethyl acetate
EtOH=ethanol
H₅IO₆ =periodic acid
LiOH=lithium hydroxide
MeOH=methanol
MgSO₄=magnesium sulfate
NaCNBH₃=sodium cyanoborohydride
Na₂CO₃=sodium carbonate
NaClO=sodium hypochlorite
NaHCO₃=sodium hydrogencarbonate
NaHPO₄=sodium hydrogenphosphate
NaHSO₃=sodium hydrogensulfite
NaOH=sodium hydroxide
Na₂WO₄.2H₂O=sodium tungstate dihydrate
NH₄Cl=ammonium chloride
Pd/C=palladium on carbon
PdCl₂(dppf)=[1,1′-bis(diphenylphosphino)ferrocene]dichloropalladium(II)
Pd(OAc)₂=palladium acetate
iPr₂EtN=diisopropylethylamine
PyBOP=Benzotriazol-1-yl-oxytripyrrolidinophosphonium hexafluorophosphate
PG=protecting group
PPh₃=triphenylphosphine
rt=room temperature
sat. aq.=saturated aqueous
SiO₂=silicon dioxide
THF=tetrahydrofuran
TiCl₄=titanium chloride
tlc=thin layer chromatography
Me=methyl
Et=ethyl

The novel compounds of the present invention can be prepared according to the following general procedures using appropriate materials and are further exemplified by the following specific examples. The compounds illustrated in the examples are not, however, to be construed as forming the only genus that is considered as the invention. The following examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

### Schemes

Compounds of the present invention can be prepared according to Scheme 1, as indicated below. Thus an α-amino ester may be added to a haloalkyl ketone to form an aminal which may be dehydrated to an imine in the presence of a dehydrating agent such as TiCl₄, MgSO₄ or isopropyl trifluoroacetate. Reduction of the imine with a reducing agent such as sodium cyanoborohydride or sodium borohydride provides the amine. A palladium-catalyzed Suzuki coupling with an appropriate boronic acid provides the left hand side of the molecule. Ester hydrolysis and coupling with the seven-membered ring amine (synthesis in J. Med. Chem. 44 1380, 2001) provides the amide. This molecule is elaborated to compounds of the present invention by removal of the amine protecting group, coupling with an aryl/heteroarylSO₂Cl or aryl/heteroarylC(O)Cl and oxidation of the alcohol to the ketone.

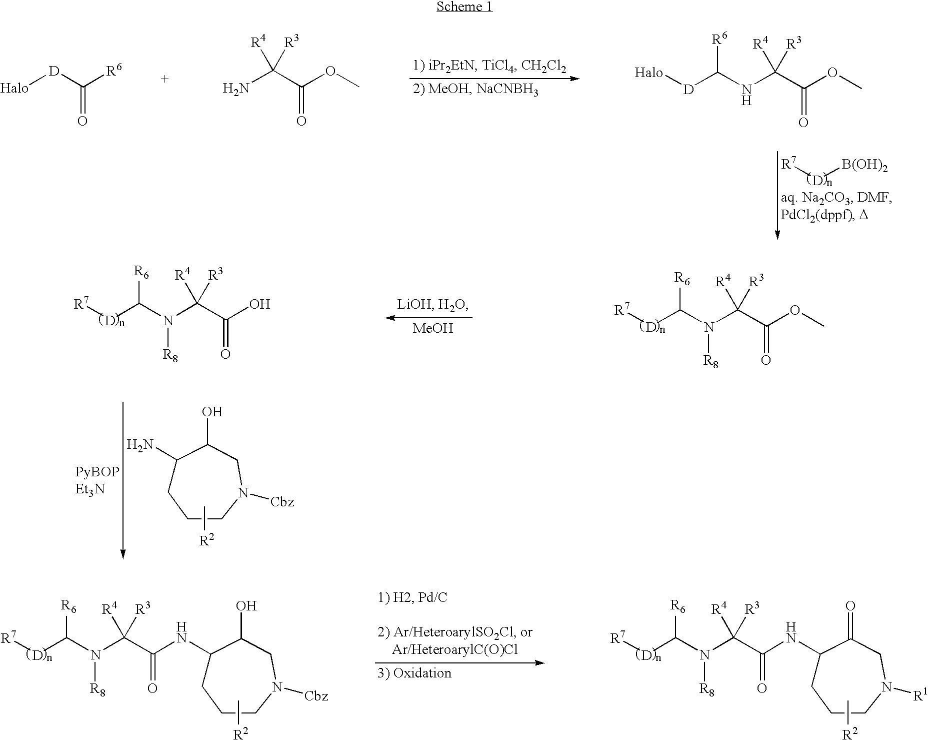

Compounds of the present invention may also be prepared according to Scheme 2, as indicated below. A haloalkylketone or aldehyde may be condensed with an amino alcohol to give a cyclic aminal. Treatment with 3 equivalents of a Grignard reagent or organolithium reagent will provide the appropriate alkylated amino alcohol. Oxidation of the alcohol with a chromium system such as H₅IO₆/CrO₃, or alternatively by a two-step oxidation (eg oxalyl chloride/DMSO/Et₃N followed by NaClO) will provide the corresponding carboxylic acid. This acid may be converted to compounds of the current invention as described in Scheme 1.

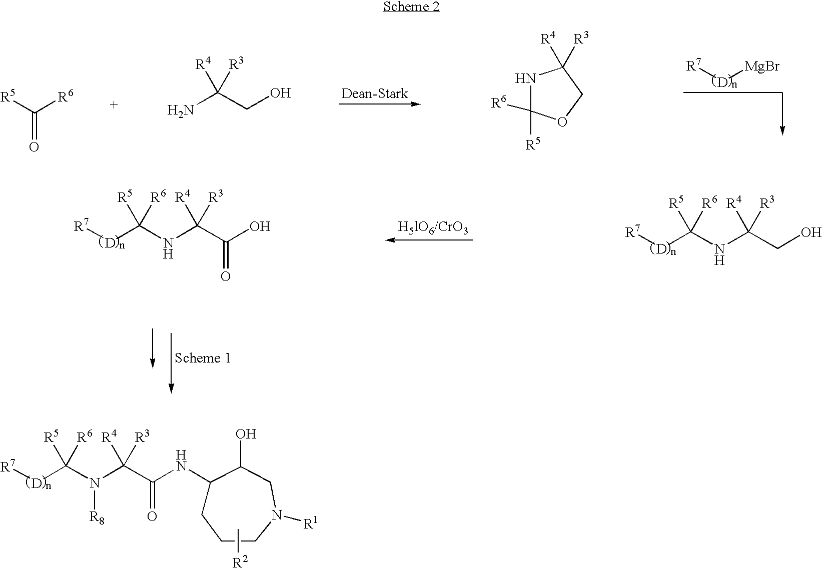

Compounds of the current invention may also be prepared according to Scheme 3, as indicated below. A hemiacetal may be condensed with an amino alcohol in which the alcohol moiety is protected with a suitable protecting group. Treatment of the resulting imine with a Grignard reagent or organolithium reagent and removal of the alcohol protecting group will provide the appropriate alkylated amino alcohol. A palladium-catalyzed Suzuki coupling with an appropriate boronic acid and oxidation of the alcohol with H₅IO₆/CrO₃ will provide the corresponding carboxylic acid. This acid may be converted to compounds of the current invention as described in Scheme 1. 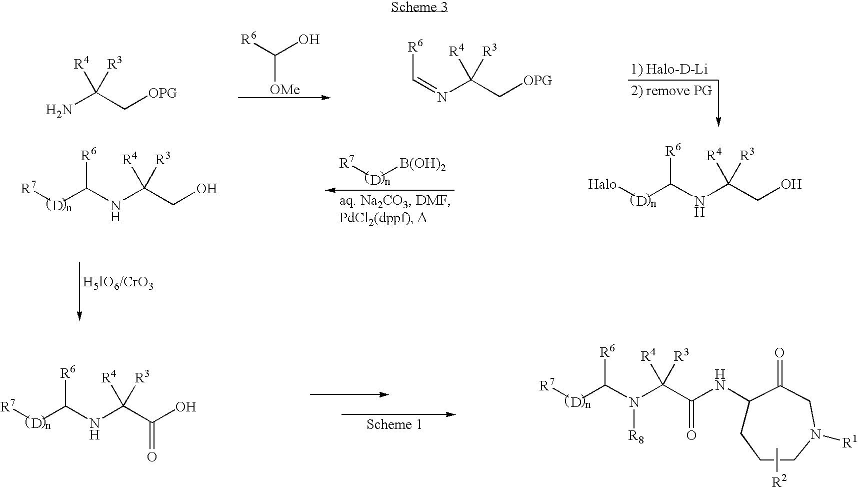
Pharmaceutical Composition

As a specific embodiment of this invention, 100 mg of N¹-(cyanomethyl)-N²-[2,2,2-trifluoro-1-(4′-piperazin-1-yl-1,1′-biphenyl-4-yl)ethyl]-L-leucinamide, is formulated with sufficient finely divided lactose to provide a total amount of 580 to 590 mg to fill a size 0, hard-gelatin capsule.

The compounds disclosed in the present application exhibited activity in the following assays.

### Cathepsin K Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; and DTT, 2.5 mM) and 25 μL of human cathepsin K (0.1 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

### Cathepsin L Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; and DTT, 2.5 mM) and 25 μL of human cathepsin L (1.5 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

### Cathepsin B Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; and DTT, 2.5 mM) and 25 μL of human cathepsin B (2.5 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

### Cathepsin S Assay

Serial dilutions (1/3) from 500 μM down to 0.0085 μM of test compounds were prepared in dimethyl sulfoxide (DMSO). Then 2 μL of DMSO from each dilution were added to 50 μL of assay buffer (MES, 50 mM (pH 5.5); EDTA, 2.5 mM; and DTT, 2.5 mM) and 25 μL of human cathepsin S (4 nM) in assay buffer solution. The assay solutions were mixed for 5-10 seconds on a shaker plate and incubated for 15 minutes at room temperature. Z-Leu-Arg-AMC (8 μM) in 25 μL of assay buffer was added to the assay solutions. Hydrolysis of the coumarin leaving group (AMC) was followed by spectrofluorometry (Exλ=355 nm; Emλ=460 nm) for 10 minutes. Percent of inhibition were calculated by fitting experimental values to standard mathematical model for dose response curve.

### EXAMPLE 1

### SYNTHESIS OF N¹-[3-OXO-1-(PYRIDIN-2-YLSULFONYL)AZEPAN-4-YL]-N²-{(1S)-2,2,2-TRIFLUORO-1-[4′-(METHYLSULFONYL)-1,1′-BIPHENYL-4-YL]ETHYL}-L-LEUCINAMIDE

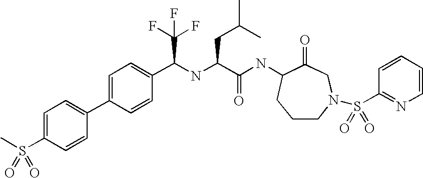

### Step 1: Preparation of (2S)-1-{[tert-butyl(dimethyl)silyl]oxy}-4-methylpentan-2-amine

To a room temperature dichloromethane (100 mL) solution of L-leucinol (6.0 g) was added triethylamine (11 mL), DMAP (0.1 g) and t-butyldimethylsilyl chloride (8.5 g). The mixture was stirred at room temperature for 2 hours and then water was added. The organic layer was separated and the aqueous further extracted with dichloromethane. The combined organic layers were washed with brine, dried with magnesium sulfate and the solvent was removed in vacuo to yield the title compound, a residue which was used as such in the next reaction. ¹H NMR (CD₃COCD₃) δ 3.48(m, 2H), 3.32(m, 1H), 2.76(m, 1H), 1.78(m, 1H), 1.22-1.02(m, 2H), 0.88(m, 15H), 0.06(s, 6H).

### Step 2: Preparation of (2S)-1-{[tert-butyl(dimethyl)silyl]oxy}-4-methyl-N-[(1E)-2,2,2-trifluoroethylidene]pentan-2-amine

A toluene (300 mL) solution of (2S)-1-{[tert-butyl(dimethyl)silyl]oxy}-4-methylpentan-2-amine from Step 1 (50 g) and tifluoroacetaldehyde methyl hemiacetal (35 mL) was heated to reflux for 16 hours during which time water was collected in a Dean-Stark trap. The solvent was evaporated in vacuum and the residue was purified on SiO₂ using hexanes and ethyl acetate (9:1) as eluant to yield the title compound. ¹H NMR (CD₃COCD₃) δ 7.88(m, 1H), 3.76-3.45(m, 3H), 1.60-1.25(m, 3H), 0.88(m, 15H), 0.06(s, 3H), 0.04(s, 3H).

### Step 3: Preparation of (2S)-2-{[(1S)-1-(4-bromophenyl)-2,2,2-trifluoroethyl]amino}-4-methylpentan-1-ol

n-Buli (2.5 M in hexanes, 42 mL) was added to a −70° C. THF (400 mL) solution of 1,4-dibromobenzene (25.8 g) and the mixture was stirred for 25 minutes. A THF (30 mL) solution of (2S)-1-{[tert-butyl(dimethyl)silyl]oxy}-4-methyl-N-[(1E)-2,2,2-trifluoroethylidene]pentan-2-amine (31 g) was then added dropwise and the mixture was stirred for 1.5 hours. It was then poured slowly into a mixture of ethyl acetate (500 mL), water (2 L), ice (300 g) and ammonium chloride (100 g) under vigorous stirring. The organic layer was separated and the aqueous further extracted with ethyl acetate (2×500 mL). The combined organic layers were washed with brine, dried with magnesium sulfate and the solvent was removed in vacuo to yield a residue, which was used as such. The residue from above was dissolved in THF (250 ml) and the solution was cooled to 0° C. A 1 M THF solution of t-butylammonium fluoride (110 mL) was added dropwise and the mixture was reacted for 4 hours. It was poured into ethyl acetate (300 mL), water (2 L) and ammonium chloride (100 g) under vigorous stirring. The organic layer was separated and the aqueous further extracted with ethyl acetate (2×100 mL). The combined organic layers were washed with brine, dried with magnesium sulfate and the solvent was removed in vacuo to yield a residue which was purified purified on SiO₂ using a gradient of ethyl acetate and hexanes (1:5 to 1:4) as eluant to yield the title compound. ¹H NMR (CD₃COCD₃) δ 7.6(2H, d), 7.45(2H, d), 4.55(1H, m), 3.65-3.7(1H, m), 3.5-3.55(1H, m), 3.25-3.35(1H, m), 2.6-2.7(1H, m), 2.25-2.35(1H, m), 1.65-1.75(1H, m), 1.3-1.4(1H, m), 1.2-1.3(1H, m), 0.75-0.9(6H, dd).

### Step 4: Preparation of (2S)-4methyl-2-({(1S)-2,2,2-trifluoro-1-[4′-(methylthio)-1,1′-biphenyl-4-yl]ethyl}amino)pentan-1-ol

A stream of nitrogen was passed through a suspension made of the bromide from Step 3 (27.7 g), 4-(methylthio)phenylboronic acid (15.7 g), 2 M Na₂CO₃ (100 mL) and n-propanol (500 mL) for 15 minutes. A 1:3 mixture (3.5 g) of Pd(OAc)₂ and PPh₃ was then added and the reaction was warmed to 70° C. and stirred under nitrogen for 8 hours. The mixture was cooled to room temperature, diluted with ethylacetate (500 mL) and poured over water (2 L) and ice (500 g). The ethyl acetate layer was separated and the aqueous further extracted with ethyl acetate (200 mL). The combined ethyl acetate extracts were washed with 0.5 N NaOH (2×200 mL), aqueous NH₄Cl, brine and dried with magnesium sulfate. Removal of the solvent left a residue that was purified by chromatography on SiO₂ using a gradient of ethyl acetate and hexanes (1:4 to 1:3) and again with acetone and toluene (1:10). The residue was dissolved in hot hexanes (200 mL) and the solution was allowed to cool to 0° C. under stirring. The obtained solid was filtered and dried to yield the title compound. ¹H NMR (CD₃COCD₃) δ 7.7(2H, d), 7.65(2H, d), 7.6(2H, d), 7.35(2H, d), 4.5-4.6(1H, m), 3.7(1H(OH), m), 3.5-3.6(1H, m), 3.3-3.4(1H, m), 2.7(1H, m), 2.5(3H, s), 2.3-2.4(1H(NH), m), 1.65-1.75(1H, m), 1.2-1.4(3H, m), 0.8-0.9(6H, dd).

### Step 5: Preparation of (2S)-4-methyl-2-({(1S)-2,2,2-trifluoro-1-[4′-(methylsulfonyl)-1.1′-biphenyl-4-yl]ethyl}amino)pentan-1-ol

To a 0° C. solution of the sulfide (19 g) from Step 4 in toluene (400 mL) was added Na₂WO₄.2H₂O (0.16 g) and Bu₄NHSO₄ (0.81 g). Then 30% hydrogen peroxide (12.2 mL) was slowly added and the mixture was stirred at room temperature for 4.5 hours. The mixture was poured slowly on a mixture of ice, dilute aqueous sodium thiosulfate and ethyl acetate. The organic layer was separated and the aqueous further extracted with ethyl acetate (2×100 mL). The combined organic layers were washed with brine, dried with magnesium sulfate and the solvent were removed in vacuo to yield a residue which was purified purified on SiO₂ using ethyl acetate and hexanes (1:1) as eluant to yield the product. ¹H NMR (CD₃COCD₃) δ 8.05(2H, d), 8.0(2H, d), 7.85(2H, d), 7.7(2H, d), 4.6-4.7(1H, m), 3.75(1H, m), 3.6(1H, m), 3.35-3.45(1H, m), 3.2(3H, s), 2.7-2.8(1H, m), 2.35-2.45(1H, m), 1.7-1.8(1H, m), 1.2-1.5(2H, m), 0.8-0.95(6H, dd).

### Step 6: Preparation of N-{(1S)-2,2,2-trifluoro-1-[4′-(methylsulfonyl)-1,1′-biphenyl-4-yl]ethyl}-L-leucine

A suspension of H₅IO₆/CrO₃ (529 mL of 0.44 M in CH3CN; see Note below) was cooled to 0° C. and a solution of the alcohol from Step 5 (20 g) in CH₃CN (230 mL) was added dropwise. The mixture was stirred at 0-5° C. for 3.5 hours. It was poured into pH 4 Na₂HPO₄ (1.5 L) under vigorous stirring and the mixture was extracted with diethyl ether (3×250 mL). The combined ether extracts were washed with water and brine (1:1), dilute aqueous NaHSO₃ and brine. The organic extract was dried with sodium sulfate, filtered and the solvents were evaporated to dryness to yield a residue that was split into two batches for the following purification.

The crude acid from above (10 g) was dissolved in isopropyl acetate (250 mL) and extracted into cold 0.1 N NaOH (3×250 mL). The combined extracts were washed with diethyl ether (250 mL) and then slowly acidified with 6 N HCl to pH 4. The carboxylic acid was extracted with isopropyl acetate (2×250 mL) and the isopropyl acetate layer dried and concentrated to yield the title compound essentially pure and used as such in the next step. Note. The oxidizing reagent (H₅IO₆/CrO₃) was prepared as described in Tetrahedron Letters 39 (1998) 5323-5326 but using HPLC grade CH₃CN (contains 0.5% water); no water was added. ¹H NMR (CD₃COCD₃) δ 8.05(2H, d), 7.95(2H, d), 7.8(2H, d), 7.65(2H, d), 4.45-4.55(1H, m), 3.55-3.6(1H, m), 3.2(3H, s), 2.8-3.0(broad m, NH/OH)1.95-2.05(1H, m), 1.55-1.6(2H, m), 0.9-1.0(6H, m).

### Step 7: Preparation of benzyl 3-hydroxy-4-[(N-{(1S)-2,2,2-trifluoro-1-[4′-(methylsulfonyl)-1,1′-biphenyl-4-yl]ethyl}-L-leucyl)amino]azepane-1-carboxylate

To a cold (0° C.), stirred solution of N-{(1S)-2,2,2-trifluoro-1-[4′-(methylsulfonyl)-1,1′-biphenyl-4-yl]ethyl}-L-leucine (605 mg, 1.37 mmol) and benzyl 4-amino-3-hydroxyazepane-1-carboxylate (326 mg, 1.23 mmol, prepared according to J. Med. Chem. 44, 1380, 2001) in DMF (10 mL) was added Et₃N (0.45 mL, 3.22 mmol). The reaction mixture was stirred for 1 h at 0° C. followed by 1 h at rt. Saturated aqueous NaHCO₃, 1 N NaOH and ether were added. The organic layer was washed with pH 3.5 phosphate buffer, dried over MgSO₄ and concentrated under reduced pressure. The residue obtained was purified by flash chromatography (gradient elution: 65% EtOAc in hexane to 100% EtOAc) to afford the title compound as a mixture of isomers.

### Step 8: Preparation of N¹-(3-hydroxyazepan-4-yl)-N²-{(1S)-2,2,2-trifluoro-1-[4′-(methylsulfonyl)-1,1′-biphenyl-4-yl]ethyl}-L-leucinamide

A stirred solution of benzyl 3-hydroxy4-[(N-{(1S)-2,2,2-trifluoro-1-[4′-(methylsulfonyl)-1,1′-biphenyl-4-yl]ethyl}-L-leucyl)amino]azepane-1-carboxylate (710 mg, 1.03 mmol) in a 2:1 mixture of EtOH/EtOAc (80 mL) was flushed with H₂ and stirred at rt for 2 h. The suspension was filtered through celite and the filtrate was concentrated under reduced pressure to afford the title compound which was used as such in the next reaction.

### Step 9: Preparation of N¹-[3-hydroxy-1-(pyridin-2-ylsulfonyl)azepan-4-yl]-N²-{(1S)-2,2,2-trifluoro-1-[4′-(methylsulfonyl)-1,1′-biphenyl-4-yl]ethyl}-L-leucinamide

To a cold (0° C.), stirred solution of N¹-(3-hydroxyazepan-4-yl)-N²-{(1S)-2,2,2-trifluoro-1-[4′-(methylsulfonyl)-1,1′-biphenyl-4-yl]ethyl}-L-leucinamide (567 mg, 1.02 mmol) in Et₃N (0.25 mL, 1.8 mmol) was added pyridine-2-sulfonyl chloride (204 mg, 1.15 mmol). The reaction mixture was warmed to rt and stirred for 1 h. The reaction was then transferred to a 4° C. fridge and left overnight. The mixture was partitioned between CH₂Cl₂ and saturated aqueous NaHCO₃. The organic layer was washed with brine, filtered through cotton and concentrated under reduced pressure. The residue was purified by flash chromatography (gradient elution: 70% EtOAc in hexane to 100% EtOAc) to afford the title compound.

### Step 10: Preparation of N¹-[3-oxo-1-(pyridin-2-ylsulfonyl)azepan-4-yl]-N²-{(1S)-2,2,2-trifluoro-1-[4′-(methylsulfonyl)-1,1′-biphenyl-4-yl]ethyl}-L-leucinamide

To a solution of N¹-[3-hydroxy-1-(pyridin-2-ylsulfonyl)azepan-4-yl]-N²-{(1S)-2,2,2-trifluoro-1-[4′-(methylsulfonyl)-1,1′-biphenyl-4-yl]ethyl}-L-leucinamide (460 mg, 0.73 mmol) in CH₂Cl₂ (15 mL) was added Dess Martin periodinane (420 mg, 0.99 mmol). The reaction was stirred at rt for 1 h and then diluted with CH₂Cl₂. The solution was washed with 1 N NaOH and brine, filtered through cotton and concentrated under reduced pressure. The residue was purified by flash chromatography (gradient elution: 60% EtOAc in hexane to 100% EtOAc) to yield the title compound.

MS (+ESI): 695.3 [M+1)⁺

What is claimed is:

1. A compound of the formula: 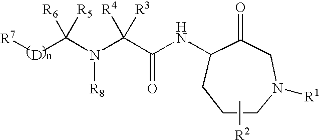
wherein R¹ is hydrogen, C₁₋₆ alkyl, —SO₂R⁹, —C(O)R⁹ or arylC₁₋₆alkyl;
R² is hydrogen, C₁₋₆ alkyl or C₃₋₆ cycloalkyl;
R³ is hydrogen, C₁₋₆ alkyl or C₂₋₆ alkenyl wherein said alkyl and alkenyl groups are optionally substituted with C₃₋₆ cycloalkyl or halo;
R⁴ is hydrogen, C₁₋₆ alkyl or C₂₋₆ alkenyl wherein said alkyl and alkenyl groups are optionally substituted with C₃₋₆ cycloalkyl or halo;
or R³ and R⁴ can be taken together with the carbon atom to which they are attached to form a C₃₋₈ cycloalkyl ring, C₅₋₈ cycloalkenyl ring, or five to seven membered heterocyclyl wherein said cycloalkyl, cycloalkenyl and heterocyclyl groups are optionally substituted with C₁₋₆ alkyl, halo, hydroxyalkyl, hydroxy, alkoxy or keto;
R⁵ is selected from hydrogen or C₁₋₆ allyl substituted with 1-6 halo;
R⁶ is aryl, heteroaryl, C₁₋₆ haloalkyl, arylalkyl or heteroarylalkyl, wherein said aryl, heteroaryl, arylalkyl and heteroarylalkyl groups are optionally substituted with halo, C₁₋₆ alkyl, C₁₋₆ haloalkyl, C₃₋₆ cycloalkyl, —SR⁹, —SR¹², —SOR⁹, —SOR¹², —SO₂R⁹, —SO₂R¹², —SO₂CH(R¹²)(R¹¹), —OR¹², —N(R¹⁰)(R¹¹) or cyano;
D is C₁₋₃ alkyl, C₂₋₃ alkenyl, C₂₋₃ alkenyl, aryl, heteroaryl, C₃₋₈ cycloalkyl or heterocyclyl wherein said aryl, heteroaryl, cycloalkyl and heterocyclyl groups, which may be monocyclic or bicyclic, are optionally substituted on either the carbon or the heteroatom with one to five substituents selected from C₁₋₆ alkyl, halo or keto;
R⁷ is hydrogen, C₁₋₆ alkyl, C₂₋₆ alkenyl, C₂₋₆ alkynyl, C₁₋₆ alkyloxy, halo, nitro, cyan heteroaryl, C₃₋₈ cycloalkyl, heterocyclyl, —C(O)OR¹⁰, —C(O)OSi[CH(CH₃)₂]₃, —OR¹⁰, —C(O)R¹⁰, —R¹⁰C(O)R⁹, —C(O)R⁹, —C(O)N(R¹²)(R¹²), —C(O)N(R¹⁰)(R¹¹), —C(R¹⁰)(R¹¹)OH, —SR¹², —SR⁹, —R¹⁰SR⁹, —R⁹, —C(R⁹)₃, —C(R¹⁰)(R¹¹)N(R⁹)₂, —NR¹⁰C(O)NR¹⁰S(O)₂R⁹, —SO₂R¹², —SO(R¹²), —SO₂R⁹, —SO₂N(R^{c})(R^{d}), —SO₂CH(R¹⁰)(R¹¹), —SO₂N(R¹⁰)C(O)(R¹²), —SO₂(R¹⁰)C(O)N(R¹²)₂, —OSO₂R¹⁰, —N(R¹⁰)(R¹¹), —N(R¹⁰)C(O)N(R¹⁰)(R⁹), —N(R¹⁰)C(O)R¹⁰, —N(R¹⁰)C(O)OR¹⁰, —N(R¹⁰)SO₂(R¹⁰), —C(R¹⁰)(R¹¹)NR¹⁰C(R¹⁰)(R¹¹)R⁹, —C(R¹⁰)(R¹¹)N(R¹⁰)R⁹, —C(R¹⁰)(R¹¹)N(R¹⁰)(R¹¹), —C(R¹⁰)(R¹¹)SC(R¹⁰)(R¹¹)R⁹, R¹⁰S—, —C(R^{a})(R^{b})NR^{a}C(R^{a})(R^{b})₂, —C(R^{a})(R^{b})N(R^{a})(R^{b}), —C(R^{a})(R^{b})C(R^{a})(R^{b})N(R^{a})(R^{b}), —C(O)C(R^{a})(R^{b})N(R^{a})(R^{b}), —C(R^{a})(R^{b})N(R^{a})C(O)R⁹, —C(O)C(R^{a})(R^{b})S(R^{a})(R^{b}) or C(R^{a})(R^{b})C(O)N(R^{a})(R^{b}); wherein said groups are optionally substituted on either the carbon or the heteroatom with one to five substituents independently selected from C₁₋₆ alkyl, halo, keto, cyano, haloalkyl, hydroxyalkyl, —OR⁹, —O(aryl), —NO₂, —NH₂, —NHS(O)₂R⁸, —R⁹SO₂R¹², SO₂R¹², SO(R¹²), SO₂N(R^{c})(R^{d}), SO₂N(R¹⁰)C(O)(R¹²), —C(R¹⁰)(R¹¹)N(R¹⁰)(R¹¹), —C(R10)(R¹¹)OH, —COOH, —C(R^{a})(R^{b})C(O)N(R^{a})(R^{b}), —N(R¹⁰)C(R¹⁰)(R¹¹), —NH(CH₂)₂OH, —NHC(O)OR¹⁰, Si(CH₃)₃, heterocycyl, aryl or heteroaryl;
R⁸ is hydrogen or C₁₋₆ alkyl;
or R⁴ and R⁸ or can be taken together with any of the atoms to which they may be attached or are between them to form a 4-10 membered heterocyclyl ring system wherein said ring system, which may be monocyclic or bicyclic, is optionally substituted with C₁₋₆ alkyl, halo, hydroxyalkyl, hydroxy, keto, OR¹⁰, SR¹⁰ or N(R¹⁰)₂;
R⁹ is selected from the group consisting of hydrogen, aryl, aryl(C₁₋₄) alkyl, heteroaryl, heteroaryl(C₁₋₄)alkyl, C₃₋₈cycloalkyl, C₃₋₈cycloalkyl(C₁₋₄)alkyl, and heterocyclyl(C₁₋₄)alkyl wherein said groups can be optionally substituted with halo or alkoxy;
R¹⁰ is hydrogen or C₁₋₆ alkyl
R¹¹ is hydrogen or C₁₋₆ alkyl;
R¹² is hydrogen or C₁₋₆ alkyl which is optionally substituted with halo, alkoxy, cyano, —NR¹⁰ or —SR¹⁰;
R^{a} is hydrogen, C₁₋₆ alkyl, (C₁₋₆ alkyl)aryl, (C₁₋₆ alkyl)hydroxyl, —O(C₁₋₆ alkyl), hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with C₁₋₆ alkyl or halo;
R^{b} is hydrogen, C₁₋₆ alkyl, (C₁₋₆ alkyl)aryl, (C₁₋₆ alkyl)hydroxyl, alkoxyl, hydroxyl, halo, aryl, heteroaryl, C₃₋₈ cycloalkyl, heterocyclyl, wherein said alkyl, aryl, heteroaryl, C₃₋₈ cycloalkyl and heterocyclyl can be optionally substituted on either the carbon or the heteroatom with C₁₋₆ alkyl or halo;
or R^{a} and R^{b} can be taken together with the carbon atom to which they are attached or are between them to form a C₃₋₈ cycloalkyl ring or C₃₋₈ heterocyclyl ring wherein said 3-8 membered ring system may be optionally substituted with C₁₋₆ alkyl and halo;
R^{c} is hydrogen or C₁₋₆ alkyl which is optionally substituted with halo or OR⁹;
R^{d} is hydrogen or C₁₋₆ alkyl which is optionally substituted with halo or OR⁹;
or R^{c} and R^{d} can be taken together with the nitrogen atom to which they are attached or are between them to form a C₃₋₈ heterocyclyl ring which is optionally substituted with C₁₋₆ alkyl, halo hydroxyalkyl, hydroxy, alkoxy or keto;
n is independently selected from an integer from zero to three;
and the pharmaceutically acceptable salts, stereoisomers and N-oxide derivatives thereof.

2. The compound of claim 2 wherein R³ and R⁴ are each independently selected from hydrogen or C₁₋₄ alkyl; or R³ and R⁴ can be taken together with the carbon atom to which they are attached to form a six membered cycloalkyl ring system, and the pharmaceutically acceptable salts, stereoisomers and N-oxide derivatives thereof.

3. The compound of claim 1 wherein R⁵ is C₁₋₆alkyl substituted with 1-6 halo and R⁶ is C₁₋₆ alkyl substituted with 1-6 halo; and the pharmaceutically acceptable salts, stereoisomers and N-oxide derivatives thereof.

4. The compound of claim 1 wherein R⁵ is hydrogen and R⁶ is C₁₋₆ alkyl substituted with 1-6 halo; and the pharmaceutically acceptable salts, stereoisomers and N-oxide derivatives thereof.

5. The compound of claim 1 wherein R⁵ is hydrogen and R⁶ is aryl or heteroaryl wherein said aryl or heteroaryl are optionally substituted with halo or —SO₂R¹²; and the pharmaceutically acceptable salts, stereoisomers and N-oxide derivatives thereof.

6. The compound of claim 1 wherein R⁴ and R⁸ or can be taken together with any of the atoms to which they may be attached or are between them to form a 4-10 membered heterocyclyl ring system wherein said ring system, which may be monocyclic or bicyclic, is optionally substituted with C₁₋₆ alkyl, halo, hydroxyalkyl, hydroxy, keto, —OR¹⁰, —SR¹⁰ or —N(R¹⁰)₂; and the pharmaceutically acceptable salts, stereoisomers and N-oxide derivatives thereof.

7. The compound of claim 6 wherein R⁴ and R⁸ can be taken together with any of the atoms to which they may be attached or are between them to form a 5 or 6 membered heterocyclyl ring system wherein said ring system, is optionally substituted with C₁₋₆ alkyl, halo, hydroxyalkyl, hydroxy, keto, —OR¹⁰, —SR¹⁰ or —N(R¹⁰)₂; and the pharmaceutically acceptable salts, stereoisomers and N-oxide derivatives thereof.

8. The compound of claim 1 selected from:
N¹-[3-Oxo-1-(pyridin-2-ylsulfonyl)azepan-4-yl]-N²-{(1S)-2,2,2-trifluoro-1-[4′-(methylsulfonyl)-1,1′-biphenyl-4-yl]ethyl}-L-leucinamide, and the pharmaceutically acceptable salts, stereoisomers and N-oxide derivatives thereof.

9. A pharmaceutical composition comprising a compound according to claim 1 and a pharmaceutically acceptable carrier.

10. A pharmaceutical composition made by combining a compound according to any one of claims 1 to 8 and a pharmaceutically acceptable carrier.

11. A process for making a pharmaceutical composition comprising combining a compound according to claim 1 and a pharmaceutically acceptable carrier.

12. A method of inhibiting cathepsin activity in a mammal in need thereof, comprising administering to the mammal a therapeutically effective amount of a compound according to claim 1.

13. The method according to claim 13 wherein the cathepsin activity is Cathepsin K activity.

14. A method of treating of preventing a disease selected from: osteoporosis, glucocorticoid induced osteoporosis, Paget's disease, abnormally increased bone turnover, periodontal disease, tooth loss, bone fractures, rheumatoid arthritis, osteoarthritis, periprosthetic osteolysis, osteogenesis imperfecta, metastatic bone disease, hypercalcemia of malignancy or multiple myeloma in a mammal in need thereof by administering to the mammal a therapeutically effective amount of a compound according to claim 1.

15. A method of treating or preventing bone loss in a mammal in need thereof by administering to the mammal a therapeutically effective amount of a compound according to claim 1.

16. A method of treating or preventing osteoporosis in a mammal in need thereof by administering to the mammal a therapeutically effective amount of a compound according to claim 1.

17. A method of treating cathepsin dependent conditions in a mammal in need thereof by administering to the mammal a therapeutically effective amount of a compound according to claim 1.

18. A pharmaceutical composition comprising a compound of claim 1 and another agent selected from: an organic bisphosphonate, an estrogen receptor modulator, an androgen receptor modulator, an inhibitor of osteoclast proton ATPase, an inhibitor of HMG-CoA reductase, an integrin receptor antagonist, or an osteoblast anabolic agent, and the pharmaceutically acceptable salts and mixtures thereof.

19. A method of treating osteoporosis comprising a compound of claim 1 and another agent selected from: an organic bisphosphonate, an estrogen receptor modulator, an androgen receptor modulator, an inhibitor of osteoclast proton ATPase, an inhibitor of HMG-CoA reductase, an integrin receptor antagonist, or an osteoblast anabolic agent, and the pharmaceutically acceptable salts and mixtures thereof.

20. A method of treating bone loss comprising a compound of claim 1 and another agent selected from: an organic bisphosphonate, an estrogen receptor modulator, an androgen receptor modulator, an inhibitor of osteoclast proton ATPase, an inhibitor of HMG-CoA reductase, an integrin receptor antagonist, or an osteoblast anabolic agent, and the pharmaceutically acceptable salts and mixtures thereof.

21. A pharmaceutical composition comprising a compound of claim 1 and another agent selected from: an organic bisphosphonate, an estrogen receptor modulator, an androgen receptor modulator, an inhibitor of osteoclast proton ATPase, an inhibitor of HMG-CoA reductase, an integrin receptor antagonist, or an osteoblast anabolic agent, and the pharmaceutically acceptable salts and mixtures thereof.

22. A compound of any one of claims 1 to 8, or a pharmaceutically acceptable salt, stereoisomer or N-oxide derivative thereof, for use in inhibiting cathepsin activity, such as cathepsin K activity.

23. Use of a compound of any one of claims 1 to 8, or a pharmaceutically acceptable salt, stereoisomer or N-oxide derivative thereof, in the manufacture of a medicament for treating or preventing a disease set forth in claim 14.
